(19) United States
(12) Patent Application Publication
(10) Pub. No.: US 2015/0323495 A1
(43) Pub. Date: Nov. 12, 2015

(54) NON-AQUEOUS MICROCHIP ELECTROPHORESIS FOR CHARACTERIZATION OF LIPID BIOMARKERS

(71) Applicant: UNIVERSITY OF NOTRE DAME DU LAC, Notre Dame, IN (US)

(72) Inventors: Larry R. Gibson, II, Mishawaka, IN (US); Paul W. Bohn, Granger, IN (US)

(73) Assignee: UNIVERSITY OF NOTRE DAME DU LAC, Notre Dame, IN (US)

(21) Appl. No.: 14/652,074

(22) PCT Filed: Dec. 20, 2013
(86) PCT No.: PCT/US2013/077134

§ 371 Date: Jun. 12, 2015

Related U.S. Application Data
(60) Provisional application No. 61/848,005, filed on Dec. 20, 2012.

(51) Int. Cl. G01N 27/447; B01L 3/00

(52) U.S. Cl. CPC G01N 27/44743; G01N 27/44791; G01N 27/44721; G01N 27/44782; B01L 3/50273; B82Y 5/00

(57) ABSTRACT

The invention provides devices and methods for the detection of hydrophobic biomarkers using 3D microchip capillary electrophoresis having a non-aqueous solvent system. Hydrophobic biomarkers can be placed in a microcapillary microchannel and electrokinetically injected into a second microcapillary microchannel through a nanocapillary array membrane. The hydrophobic biomarkers can then be separated and analyzed via mass spectrometry. Certain hydrophobic biomarkers can indicate a particular disease state.

### RELATED APPLICATIONS

This application claims priority under 35 U.S.C. §119(e) to U.S. Provisional Patent Application No. 61/848,005, filed Dec. 20, 2012, which is incorporated herein by reference.

### GOVERNMENT SUPPORT

This invention was made with government support under Grant No. DBI-0852741 awarded by the National Science Foundation. The government has certain rights in the invention.

### BACKGROUND OF THE INVENTION

Reactive oxygen species (ROS), produced by routine metabolic events in aerobic organisms, pose a threat to cellular components, including proteins, lipids, and DNA when overproduced. The exact cause of ROS overproduction is not yet fully elucidated, partially because these harmful molecules display extremely short half-lives. One alternative is to analyze the degradative products of ROS-induced oxidation. The presence of these biomarkers has been successfully correlated to connect oxidative stress with a number of debilitating conditions.

Phospholipids are particularly susceptible to ROS. More specifically, peroxidation of phospholipid arachidonyl residues by ROS generates prostaglandins, a complex group of biomarkers found in biofluids. Isoprostanes, a subset of prostaglandins, have been utilized as indicators of oxidative stress in cardiovascular disease, asthma, hepatic sclerosis, scleroderma, and Alzheimer's Disease (AD).

The concentration of fluid-borne phospholipids and the associated products of lipid peroxidation are a good indicator of the extent of damage brought about by ROS. However, determinations of isoprostanes are typically performed using commercial immunoassay kits, which despite picomolar limits of detection, are relatively expensive and can be slow. Thus, diagnostics capable of rapidly processing patient biofluids to resolve their complex molecular composition are needed.

Capillary electrophoresis (CE), a powerful separation technique, has been used to process DNA, RNA, protein, peptides, and metabolite mixtures in minutes, using nanoliters of biological sample, and microliters of low cost separation buffer. Despite such minute quantities of material required, direct processing of patient biofluids via CE is very challenging due to the vast number of distinct molecular entities involved. Unfortunately, neither conventional CE nor microchip electrophoresis (MCE) is well-suited for lipid determinations, because common separation buffers consist of inorganic salts in aqueous media, in which lipids tend to aggregate. One solution, micellar electrokinetic chromatography (MEKC), affords the ability to resolve molecules based not only on their electrophoretic mobility, but also their hydrophobicity, is well-suited to lipid analysis, and has been used to analyze hydrophobic mixtures in both capillaries and microchips. However, MEKC cannot be directly coupled to mass spectrometric detection because it requires high (e.g., mM) concentrations of surfactant, resulting in analyte signal suppression and contamination by separation additives. Another promising alternative, non-aqueous capillary electrophoresis (NACE), exploits increased solubility of hydrophobic analytes in organic separation solvents and has been applied to characterize biomarkers and pharmaceutical compounds.

Organic solvents used in NACE separations, coupled with inorganic background electrolytes (BGEs) such as NaCl, phosphates, and borate, have produced high efficiency separations. Unfortunately, the concentrations of some of these BGE/solvent solutions required for electrophoretic separations have been reported in the high millimolar range, thereby hampering detection and proper analysis.

Therefore, the identification of specific and easily measured hydrophobic biomarkers, particularly lipid biomarkers, will have a significant impact on ROS-induced disease diagnosis and treatment. Accordingly, what is needed is a robust, simple, accurate and cost effective device and method to identify lipid biomarkers indicative of a disease state.

### SUMMARY

The invention provides a 3-D microfluidic device and methods for detecting lipid biomarkers using the 3-D microfluidic device. In one embodiment, the invention provides a device for isolating lipid biomarkers from a bodily fluid. The device can include a 3-D microfluidic device having a first layer, second layer and third layer where the first layer has a main microchannel that extends the length of the slab, which comprises the layers of the device. This channel can also be fabricated in a serpentine pattern to accommodate longer distances, as desired. The main microchannel can have a main microchannel ending at each end. The second layer has at least one sample loading microchannel that extends a certain distance within the second layer where the sample loading microchannel has a first and second sample loading microchannel endings, where the sample loading microchannel is transverse to the main microchannel. The third layer can be a nanocapillary array membrane that is disposed between the main microchannel and sample loading microchannel, where the nanocapillary array membrane allows the main microchannel and sample loading microchannel to be in fluid communication.

In one embodiment, at least one tertiary microchannel intersects a main microchannel within the first layer.

In some embodiments, there is a plurality of main microchannels in the first layer.

In some embodiments, there is a plurality of cross microchannels in the second layer, each having a first end and a second end.

In some embodiments, the main microchannel is coupled to a mass spectrometer device.

In some embodiments, the nanocapillary array membrane is about 6 μm to about 10 μm thick and has pores of about 10 nm to about 2000 nm, or about 50 nm to about 500 nm, or about 95 nm and about 105 nm in diameter.

In another embodiment, the invention provides for a method for detecting lipid biomarkers from a bodily fluid using a 3-D microfluidic device, the method comprising:

adding a non-aqueous solvent to a 3-D microfluidic device, injecting a biological sample into a sample loading microchannel and applying a voltage so that the sample in injected through the nanocapillary array membrane and into the main microchannel. A second voltage applied to the main microchannel can be used to drive electrophoretic separation of the sample where the main microchannel is coupled to a detection device, and the sample can be analyzed to identify a lipid biomarker indicative of a disease state.

In some embodiments, the non-aqueous solvent includes N-methylformamide (NMF). In various embodiment, the non-aqueous solvent may optionally include at least one tetraalkylammonium salt. In additional embodiments, the detection of the sample is free of a synthetic label. In further embodiments, the detection device is mass spectrometer.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Exploded view of a 3-D microfluidic separation device.

FIG. 2. Schematic representation of a 3-D microfluidic device having a fused silica capillary and embedded metallic electrical wire for coupling the microfluidic device to a mass spectrometry device for analysis.

FIG. 3. Schematic depiction of the flow of analytes during use of the microfluidic device; A) Addition of sample; B) Initial lateral flow of sample; C) Vertical electrophoretic injection of sample; and D) Lateral electroosmotic transport and sample separation.

FIG. 4. Top view fluorescence images depicting gated injection of a phospholipid (10 μM NBD-PA in 1 mM TBA-TPhB/NMF) from a vertical microchannel to a horizontal microchannel across an array of 100 nm pores, and the corresponding driving potential configuration at each stage: (A) before injection (t=0 s), (B) after electrophoretic injection (Vᵢₙⱼ=10 V, t=1 s), and (C) at the onset of separation where transport downstream moves from right to left (Vₛₑₚ=900 V, t=3 s). B, BW, S, and SW represent the buffer, buffer waste, sample, and sample waste reservoir assignments for each of the 3 stages of operation.

FIG. 5. Electropherograms depicting how the duration, Δtᵢₙⱼ, and voltage magnitude, ΔVᵢₙⱼ, of gated injection influence the lipid (10 μM NBD-PA in 10 mM TBA-TPhB/NMF) band observed 400 μm downstream in the separation microchannel. (A) Series of NBD-PA bands injected at 50 V for 1 s, 3 s, 5 s and 10 s. (B) Series of NBD-PA bands injected for 1 s at 10 V, 50 V, 100 V, and 200 V. In both experiments, Eₛₑₚ=212 V cm⁻¹.

FIG. 6. Electropherograms illustrating the relationship between dispersion of injected lipid (10 μM NBD-PA in 100 μM TBA-TPhB/NMF solution) bands and the magnitude of the electric field driving separation. ΔVᵢₙⱼ=10 V and Δtᵢₙⱼ=1 s.

FIG. 7. Electropherogram demonstrating high-resolution lipid separation via NAME. Peaks are observed 3.5 cm downstream of the injection point. (A) Electrophoretic separation of a binary analyte mixture: 10 μM NBD-PA (1) and NBD-PG (2) in 100 μM TBA-TPhB/NMF. (B) Electrophoretic separation of a ternary analyte mixture: 10 μM NBD-PA (1), NBD-PG (2), and CoA (3) in 100 μM TBA-TPhB/NMF. ΔVᵢₙⱼ=50 V, Δtᵢₙⱼ=1 s, and Eₛₑₚ=424 V cm⁻¹.

FIG. 8. Electropherograms demonstrating the sensitivity of the EMCCD for injected analytes from reservoirs with initial concentrations: 100 pM (A), 1 nM (B), 10 nM (C), 100 nM (D), and 1 μM (E).

FIG. 9. The relationship between SNR and the NBD-PA (lipid) concentration (C).

FIG. 10. Relationship between the average total ion signal of the mass spectrometer and the inlet (capillary) temperature of the mass spectrometer.

FIG. 11. Dependence of the total ion signal of the mass spectrometer on the electroosmotic flow supplied to the nanospray ionization source on the device.

FIG. 12. Mass spectrum of a lipid introduced to the mass spectrometer directly from the device via nanospray ionization.

### DETAILED DESCRIPTION

Over production of reactive oxygen species (ROS) can cause damage to cellular components such as proteins, nucleic acids and lipids. This is known as oxidative stress. Interaction of ROSs with lipids causes lipid peroxidation, leading to the formation of prostaglandins, a useful biomarker for oxidative stress.

Current methods for detecting lipid peroxidation products include immunoassay kits, conventional capillary electrophoresis, microchip electrophoresis, micellar electrokinetic chromatography and non-aqueous capillary electrophoresis. Unfortunately, each of these methods has disadvantages such as aggregating lipids or requiring the addition of a surfactant that can interfere with downstream analysis. Therefore, there is a need for a device and method to overcome these difficulties.

Described herein is the disclosure of devices and methods of using three dimensional non-aqueous capillary electrophoresis to identify hydrophobic biomarkers without causing aggregation of the target analytes. The methods are compatible with sensitive downstream analysis such as mass spectrometry.

Capillary electrophoresis (CE) relies on the movement of ions through a thin capillary tube, typically made of silica, under the influence of an applied electric field. Ions of opposite charge to electrodes on either end of the voltage will migrate toward that electrode. Thus, ions that are negatively charged will move or migrate toward the positively charged electrode and vice versa for the positively charged ions. This is known as “electrophoretic mobility.” CE is a powerful tool because each ion will migrate at a different rate with high resolution, due to the ion's quantity of charge compared to its relative hydrodynamic size and charge-to-mass ratio. The actual mobility of an ion takes into account the environment in which the ion exists in during CE. For example, electrophoretic mobility will differ from actual mobility when viscosity changes and different voltages are applied. Ions can also move under the influence of “electro-osmotic flow”, which occurs when a negative charge on the inner glass surface of the capillary produces a bulk flow of liquid towards the cathode, enabling the migration and detection of uncharged ligands.

A typical CE apparatus includes a cathode, an anode, a high voltage power supply, and a non-aqueous solvent that fills the capillary and is present in non-aqueous solvent chambers at each end of the capillary. The anode and cathode are immersed in the two solvent chambers along with the capillary ends. The apparatus also includes a detector and a data output and handling device.

Samples can be introduced into the capillary by two different methods. Electrokinetic injection can be used to introduce analytes carrying an electric charge and is accomplished by placing one end of the capillary into the sample to be injected and briefly applying an electric field. Under these conditions, the sample analyte(s) migrate into the capillary based on their electrophoretic mobility. Hydrodynamic injection is a more general method and requires the application of pressure or a vacuum to one end of the capillary. The pressure differential between the two opposite ends of the capillary introduces the analyte into the capillary for subsequent electrophoretic analysis.

Once injected, the migration of the analytes is then initiated by an electric field that is applied between the non-aqueous solvent chambers at each end of the capillary and is supplied to the electrodes by the high-voltage power supply. The direction of electrophoresis can be either from the anode (injection end) to the cathode (outlet end), or vice versa, depending on the charge of the analyte. If sufficient electroosmotic flow is present, all ions, positive or negative, migrate through the capillary in the same direction from the anode (injection end) to the cathode (outlet end). The analytes separate as they migrate due to differences in their mobility and are detected near the outlet end of the capillary. The output of the detector is sent to a data output and handling device such as an integrator or computer. The data is then displayed as an electropherogram, which reports detector response as a function of time. Separated entities can appear as peaks with different migration times, peak shapes, and peak areas in an electropherogram.

Analytes separated by CE can be detected by UV, UV-Vis absorbance, or fluorescence (natural fluorescence, chemical modification to introduce fluorescent tags, or laser-induced fluorescence). Preferably, CE may be directly coupled to a mass spectrometer. For this purpose, the capillary outlet serves as a nanospray ionization source. The resulting ions can then be analyzed by a mass spectrometer.

### Lipid Oxidation and Biomarkers.

Lipids are the primary components of biological membranes. The geometry of the lipids determine a number of membrane properties including fluidity, permeability, and formation of microdomains. In addition to this passive structural role, lipids actively participate in cell signaling by acting 1) as precursors for signaling molecules and 2) by directly interacting with proteins. For example, phosphatidylinositol-4,5-bisphosphate generates several molecules important in second messenger systems. Enzyme mediated hydrolysis of phosphatidylinositol-4,5-bisphosphate yields diacylglycerol, a precursor to the signaling lipid phosphatidic acid which has been tied to signaling pathways involved in cell growth, proliferation, reproduction, and hormone response. Another important metabolite created from phosphatidylinositol-4,5-bisphosphate includes the fatty acid arachidonic acid, the precursor of eicosanoids, a signaling lipid that plays a role in inflammatory processes. Other important lipid signaling molecules include sphingolipids and ceramides. The lipid molecules derived from these lipids act to control cellular proliferation, differentiation, and apoptosis. Finally, in addition acting as signaling molecules or precursors, membrane lipids can also be a part of the signal transduction pathway by forming complex lipid-protein and protein-protein interactions. As an example, the phosphoinositides interact with a variety of different proteins to regulate cellular functions such as calcium levels and membrane transport.

Given the abundance and diversity of lipids in serum and other biological fluids, together with the diverse roles these molecules play, it is no surprise that various lipids have been found to be biomarkers of disease. One well-recognized example is the study of lipid oxidation products caused by reactive oxygen species (ROS), which causes cell damage by reacting with proteins, nucleic acids or lipids. Oxidative damage accumulates when natural antioxidant defense mechanisms are inadequate to deal with the amount of ROS present. The resulting cell damage can result in any number of diseases. Oxidative damage is known to be a primary or secondary mechanism in a number of diseases, including atherosclerosis, cancer, cardiovascular disease, diabetes, rheumatoid arthritis, and chronic liver disease. The oxidation products of lipids can be used as an indicator of oxidative stress, with one well-established example being isoprostanes, prostaglandin-like structures formed from the oxidation of fatty acids by ROS. Also, oxidized versions of the common molecules glycerophosphocholine and cholesterol are strongly associated with atherosclerotic lesions. Finally, some oxidized phospholipids are strongly associated with the induction of cell death via apoptosis.

Additionally, the role of lipids in signaling processes makes them attractive biomarkers and targets in the study of cancer particularly. Lipid metabolites such as those discussed above are produced in response to the appropriate cell signals and are therefore early indicators of pathway activation. It is likely that acute or chronic perturbations of the levels of these signaling molecules will correlate with some emerging pathology. For example, the extent of phosphorylation of glycerophosphoinositides and associated downstream pathways play an important role in cell cycle regulation and cell death, making this lipid a molecule of interest in understanding cancer pathways. Also, alterations in the levels of sphingolipids are associated with a number of cancer types. Furthermore, higher levels of ceramides, signaling molecules derived from these lipids, are associated with apoptosis while sphingosine-1-phosphate is associated with cell growth and metastasis.

The eicosanoid family of lipids includes prostacyclins, thromboxanes, prostaglandins, leukotrienes and epoxyeicosatrienoic acids. Eicosanoids are local signaling molecules having various roles in inflammation, fever, regulation of blood pressure, blood clotting, immune system modulation, control of reproductive processes and tissue growth, and regulation of the sleep/wake cycle.

Oxidation of the phospholipid arachidonyl residues produces prostaglandins. Isoprostanes are a subset of prostaglandins containing a prostane (cyclopentane) ring. These are further divided into different subclasses labeled A-K, the most abundant of these being the A, E, F, H and J subtypes. Isoprostanes and derivatives are associated with many diseases including but not limited to cardiovascular disease, asthma, hepatic sclerosis, scleroderma, Rheumatoid Arthritis and Alzheimer's disease. Moreover, members of the eicosanoid family can be used as biomarkers, for example, for Parkinson's Disease, Multiple Sclerosis, Lou Gehrig's Disease, Atherosclerosis, Lupus, Erythematosus, Niemann Pick type C, COPD, interstitial lung disease, cystic fibrosis (CF), acute respiratory distress syndrome (ARDS), pulmonary sarcoidosis and obstructive sleep apnea.

The hydrophobic biomarkers, including lipid biomarkers, can be taken from any bodily fluid. Preferably, the bodily fluid is taken from the blood, plasma, saliva, breath condensate or urine. One skilled in the art will recognize that a biological sample can also be taken from, but not limited to the following bodily fluids: peripheral blood, ascites, cerebrospinal fluid (CSF), sputum, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation that may be of fetal or maternal origin. The biological sample may also be a tissue sample or biopsy that may contain lipid peroxidation products.

### 3-D Microfluidic Devices.

The present invention generally provides 3-D microfluidic devices, as well as methods of using these devices in the analysis of hydrophobic biomarkers from fluid borne materials that is simple, quick, highly accurate, repeatable, easily transportable and cost effective. The hydrophobic biomarkers can include, but are not limited to lipids, hydrophobic peptides, hydrophobic amino acids, glycoproteins, nucleosides, DNA adducts, proteoglycans, carbohydrates or another biomarkers or metabolites thereof capable of being solvated in a non-aqueous capillary electrophoresis device described herein.

It should be noted that in some embodiments, the 3-D microfluidic device described as having layers though the device may be a singular seamless device, where layers refer to functional areas and on specifically discrete layers. Alternatively, the same 3-D microfluidic device can be described as having multiple horizontal planes even though the device itself is a singular seamless device. The use of layer terminology, as would be recognized by one of skill in the art, serves to simplify the description of the device.

The 3-D microfluidic devices of the invention can include a multi-layer central body structure in which the various microfluidic elements are disposed. The body structures of the microfluidic devices typically employ a solid or semi-solid substrate that is typically planar in structure, i.e., substantially flat or having at least one flat surface. Suitable substrates may be fabricated from any one of a variety of materials, or combinations of materials that are compatible with the non-aqueous solvents, background electrolytes and voltage ranges contemplated herein. Often, the planar substrates are manufactured using solid substrates common in the fields of microfabrication, e.g., silica-based substrates, such as glass, quartz, silicon or polysilicon, as well as other known substrates, i.e., gallium arsenide. In the case of these substrates, common microfabrication techniques, such as photolithographic techniques, wet chemical etching, micromachining, i.e., drilling, milling and the like, may be readily applied in the fabrication of microfluidic devices and substrates. Alternatively, polymeric substrate materials may be used to fabricate the devices, including, e.g., polydimethylsiloxanes (PDMS), polymethylmethacrylate (PMMA), polyurethane, polyvinylchloride (PVC), polystyrene polysulfone, polycarbonate, polymethylpentene, polypropylene, polyethylene, polyvinylidine fluoride, ABS (acrylonitrile-butadiene-styrene copolymer), thermoplastic elastomers and the like. In the case of such polymeric materials, injection molding or embossing methods may be used to form the substrates having the microchannel and reservoir geometries as described herein. In such cases, original molds may be fabricated using any of the above described materials and methods. The reservoirs, wells and microchannels can be fabricated into or on the 3-D microfluidic device using methods known to the skilled artisan.

The 3-D microfluidic device contains at least one main microchannel or separation microchannel and at least one sample loading microchannel or cross microchannel. The width of the aforementioned microchannels can be appropriately set according to the size, purpose of use, etc. of the microchip. Specifically, it may be desirable, from the viewpoint of obtaining sufficient analytical sensitivity, that the width of the aforementioned microchannel is 0.1 μm or more, preferably 10 μm or more. In addition, it may be desirable, from the viewpoint of sufficient analytical accuracy, that the width of the aforementioned microchannel is about 150 μm or less, preferably about 100 μm. Further, although the length of the aforementioned separation microchannel can be appropriately set according to the size of the 3-D microfluidic device and the compound to be analyzed, it may be desirable that the effective length is longer to achieve optimal separation. It may be desirable, from the viewpoint of obtaining sufficient resolution, that the length is about 10 mm to about 50 mm.

In certain embodiments, the 3-D microfluidic device optionally includes microchannels that have narrower width dimensions, particularly at the injection point of the device. In particular, by narrowing the dimensions at least at the injection intersection, one can substantially reduce the size of the sample that is injected into the analysis microchannel, thereby providing a narrower band to detect, and thus, greater resolution between adjacent bands.

Moreover, in some embodiments, the 3-D microfluidic device can have at least one tertiary microchannel that is not a main microchannel or loading microchannel. The tertiary microchannel can be in the first layer or second layer and may intersect a main microchannel or sample loading microchannel. The tertiary microchannel can have the same dimensions of the main microchannel or sample loading microchannel, or in some embodiments, may differ from those microchannels.

The 3-D microfluidic device also contains at least one reservoir or microchannel endings. The size of the reservoir can be appropriately set according to the sample volume. Specifically, it is desirable, from the viewpoints of handling during sample introduction and electrode thickness, that the diameter of the reservoir is about 0.05 mm or more, preferably about 1 mm or more, and it may be desirable from the viewpoint of the amount of sample used that the diameter is about 5 mm or less, preferably about 3 mm or less, or more preferably 1 mm. In some embodiments, there is a plurality of reservoirs or microchannel endings, for example, about 2-4 reservoirs, about 2-6 reservoirs, about 2-8 reservoirs, about 2-10 reservoirs, or about 2-12 reservoirs. Each of the aforementioned reservoirs can be connected to the sample loading microchannel or main microchannel as required.

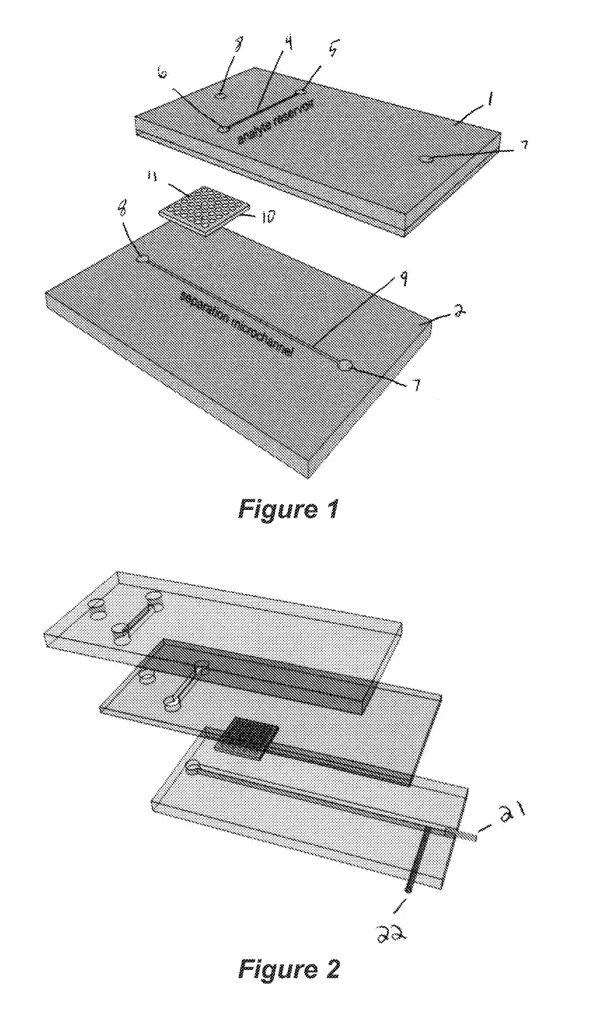

FIG. 1 refers to an exemplary embodiment of the 3-D microfluidic device. The 3-D microfluidic devices described herein can contain at least three layers (planes). A first layer (2) contains at least one main microchannel or separation microchannel (9). A second layer (1) contains at least one sample loading microchannel (4). A third layer contains at least one nanocapillary array membrane (NCAM) (10) that is disposed between the first and second layers where the NCAM allows the first and second layer to be in fluid communication.

The first layer (2) contains at least one main microchannel (9) that is disposed within the substrate through which samples are transported and subjected to a particular analysis. The first layer (2) can also contain a plurality of main microchannels. Preferably, the main microchannel (9) is substantially linear or straight, without bends. Other embodiments of the device can have serpentine microchannels or microchannels that bend at an angle or any combination thereof. Moreover, some embodiments may have more than one main microchannel or may have at least one cross microchannel that intersects the main microchannel in the first layer. The main microchannel has at least one main microchannel ending or reservoirs (7), (8) located at the end of the main microchannel (9).

The second layer (1) contains at least one sample loading microchannel (4) that is in a different plane relative to the first layer (2) where the at least one sample loading microchannel (4) is transverse to the main microchannel (9) but does not intersect the main microchannel (9). The sample loading microchannel (4) contains at least one reservoir or sample loading microchannel endings (5), (6) at each end of the sample loading microchannel (4). The device may contain a plurality of sample loading microchannels that are substantially straight but may also have bends or angle or a combination thereof disposed in the second layer. The plurality of sample loading microchannels may be transverse to the main microchannel. Each of the plurality of sample loading microchannels has at least one sample microchannel ending at one end, and preferably at both ends.

In some embodiments, the shapes of the liquid reservoirs or microchannel endings are substantially cylindrical. However, the shapes of the liquid reservoirs or microchannel endings are not particularly limited as long as they do not cause any problems in introduction and recovery of the sample described later. For example, each of reservoirs or microchannel endings may have an arbitrary shape, such as a quadrangular prism shape, a quadrangular pyramidal shape, a conical shape, or a shape formed by combining them. Furthermore, the volumes and shapes of the liquid reservoirs or microchannel endings may be identical to or different from one another.

The third layer of the microchip device is a NCAM (10). The NCAM layer (10) is disposed between the first layer (2) and the second layer (1). The NCAM is made of a suitable material known to the skilled artisan, but preferably is made of polycarbonate, polymethylacrylate, metal oxides (e.g. aluminum oxide) or other material compatible with a non-aqueous solvent. The NCAM can also have a coating made of the same material or different than the body of the NCAM. In one aspect, the coating of an NCAM is a polyester. The NCAM is preferably about 1-100 μm thick, more preferably about 1-15 μm thick, and more preferably about 6-10 μm thick. The NCAM capillary array can have a pore density of about 3×10⁸ cm-² to about 6×10⁸ cm⁻², or more preferably about 4×10⁸ cm⁻². The diameter of the pores within this array ranges from 10 nm to 10 μm (Sickman et al., J. Chromatogr. B 2002, 771(1-2), 167-196). The NCAM allows the main microchannel (9) and the sample loading microchannel (4) to be in fluid communication and is the site of electrokinetic injection of the sample from the sample loading microchannel to the main microchannel. The NCAM prevents essentially all sample diffusion from the sample loading microchannel to the main microchannel prior to electrokinetic injection. During electrokinetic injection, the NCAM acts as an electrically active gate to allow a specified amount of sample to pass from the sample loading microchannel to the main microchannel. After electrokinetic injection, the NCAM again prevents essentially all diffusion from the sample loading microchannel to the main microchannel.

The microchannels and reservoirs of the 3-D microfluidic device are ideally filled with an electrophoresis solvent that is compatible with the target biomarker (e.g., a lipid, hydrophobic peptide, etc.). The particular solvent conditions appropriate for a specific target may be determined by experimentation according to methods well known to those of ordinary skill in the art. The electrophoresis solvent is preferably non-aqueous in order to adequately solvate the hydrophobic biomarkers and to allow the appropriate downstream detection and analysis of the lipid biomarkers. Moreover, the solvent must be compatible with the substrate used to fabricate the 3-D microfluidic device. The solvent can be, but is not limited to, methanol, ethanol, acetonitrile, formamide, dimethylformamide (DMF), N-methylformamide (NMF), dimethylsulfoxide (DMSO), phenol, tert-butyl alcohol, tetrahydrofuran, sulfonic acid, acetic acid, pyridine, tetrachloromethane, 1,2-dichloroethane, acetone, nitrobenzene, benzene, or a combination thereof.

The solvent can also contain a background electrolyte to provide a vehicle for electro-osmotic flow. The choice of background electrolyte must be soluble in the non-aqueous solvent. Electrolytes can include, but are not limited to, magnesium acetate, sodium chloride, phosphates, borate, ammonium chloride, acetic acid, trifluoroacetic acid, formic acid, methane sulfonic acid, sodium acetate and tetraalkylammonium salts. In preferred embodiments, tetraalkylammonium salts such as tetrabutylammonium tetraphenylborate (TBA-TPhB) and tetraphenylphosphonium tetraphenylborate (TPhP-TPhB) are used as the background electrolyte. These electrolytes are especially desirable when the downstream detection method is via a mass spectrometry device because they will not suppress the sensitivity of the mass spectroscopy device. The concentration of background electrolyte can be 0 μM (i.e., absent), about 0 μM to about 10 mM, preferably about 0 μM to about 1 mM, and more preferably about 100 μM. Preferably, the pH of the non-aqueous system is about 8 to about 13.

Because capillary electrophoresis is a microscale technique, only small amounts of hydrophobic biomarkers are required for screening. In contrast, alternative techniques such as NMR and isothermal calorimetry can consume large amounts of biological material. In preferred embodiments, samples as small as 1 nL can be used for an electrophoretic assay run. The concentration of target compound can range, for example, from about 1 femtomolar to about 1 micromolar.

The 3-D microchip device can include one or more electrodes that are operably coupled with the substrate and microchannels. The electrodes can be located within a reservoir or electrode space that is fluidly coupled with a microchannel. A preferred embodiment of contains a plurality of electrodes, preferably about 2-4 electrodes, about 2-6 electrodes, about 2-8 electrodes, about 2-10 electrodes, or about 2-12 electrodes.

The components can also include electrophoretic electrodes that can be removable or coupled with a microchannel structure microchip body. The electrophoretic electrodes can be operably coupled with each opening end of the microchannel. The electrophoretic electrodes can include an anode and a cathode that can be separated by the microchannel with either electrode being at either opening. In one aspect, the anode is located at an entrance of the microchannel, and the cathode is located at the exit of the microchannel.

The electrodes can also be affixed in the substrate body with adhesive, such as but not limited to, acrylic adhesive, silicone adhesives, isobutylene adhesives or other contact adhesives or other fixing agent, such as but not limited to, tape, glue, friction, or others. Also, a fluid tight seal can be used to hold the electrodes in the device body. Optionally, the electrodes are fabricated into the 3-D microfluidic device.

The electrodes can be operably coupled to a power supply or with a computing system, or both. The computing system can be configured for receiving electronic data from the electrodes. Also, the computing system can be configured for receiving and/or transmitting electronic data with the electrophoresis electrodes. The computing system can have data computing components comprising code for executable instructions for operating with the one or more electrodes by, for example, modulating properties of electronic flow between the electrophoresis electrodes; determining properties of electronic flow between the electrophoresis electrodes; performing voltometry, conductometry, amperometry or potentiometry or combinations thereof; receiving and/or recording data for voltometry, conductometry, amperometry or potentiometry or combinations thereof; or the like. The computing system can also provide instructions that include obtaining measurements of voltometry, conductometry, amperometry or potentiometry or combinations thereof.

Detection of lipid biomarkers can be achieved by a number of methods including UV-Vis absorbance or fluorescence (natural fluorescence, chemical modification to introduce fluorescent tags or laser-induced fluorescence) and by mass spectrometry.

In some embodiments, mass spectrometry can be used to identify lipid biomarkers. This method is desirable because of the accuracy, sensitivity and small sample requirements needed for mass spectrometry. Moreover, the use of a non-aqueous solvent such as NMF with small amounts of a background electrolyte (e.g. TBA-TPhB or TPhP-TPhB) are compatible with mass spectrometry and do not suppress or degrade signal detection and identification. Furthermore, mass spectroscopy does not require the addition of a label or modification of the target lipid biomarkers for detection and identification.

Mass spectrometric analysis can produce a record of the masses of the atoms or molecules in a sample material. Mass spectrometry detects ionized chemical or biological compounds to produce charged fragments, (ions) which are then separated by their resulting mass-to-charge ratio.

Generally, mass spectrometry has three parts: an ion source, a mass analyzer and a detector. The ionizer converts the sample or a portion thereof into ions. Many different ionization techniques exist and can be adapted to the type of sample (e.g. phase of the sample) and the efficiency of ionization of the sample. Types of ionization include, but are not limited to electronic ionization, chemical ionization, electrospray ionization, matrix assisted laser desorption/ionization (MALDI), inductive coupling plasma sources, spark ionization and thermal ionization (TIMS).

The ions are then sent to a mass analyzer. The mass analyzer generally contains an electric and magnetic field that interact with the ionized sample. The speed and direction of the ions is affected by the mass-to-charge ration as they interact with the electric and magnetic fields. Types of mass analyzers can include, but are not limited to sector field, time-of-flight (TOF) quadrupole mass analyzer/filter, quadrupole trap, and Fourier transform mass spectrometry (FTMS).

The spectrum of ions is then collected by the detector, which records the abundance of each type of ion and gives a mass spectrum analysis to the end user. Types of detectors include, but are not limited to tandem mass spectroscopy, gas chromatography mass spectroscopy (GC-MS), liquid chromatography mass spectroscopy (LC-MS), and ion mobility mass spectroscopy.

Coupling of the 3-D microfluidic device can be achieved by adding a capillary outlet to introduce the lipid biomarkers into an ion source that utilizes nanospray ionization. The resulting ions are then analyzed by the mass spectrometer. FIG. 2 show an embodiment substantially similar to the device of FIG. 1, but which is modified for use with a mass spectrometer. Connected to the main microchannel fluid reservoir is a capillary (21), such as a fused silica capillary, and an electrode (22), for configuring the device with a mass spectrometer. The electrode (22) can be, for example, a metal wire, a thin layer electrode, or a conductive fluid. The electrode (22) serves to drive the electroosmotic fluid flow required for upstream electrophoretic separations, and provides the ionization source with a sufficiently high voltage to perform nanospray ionization.

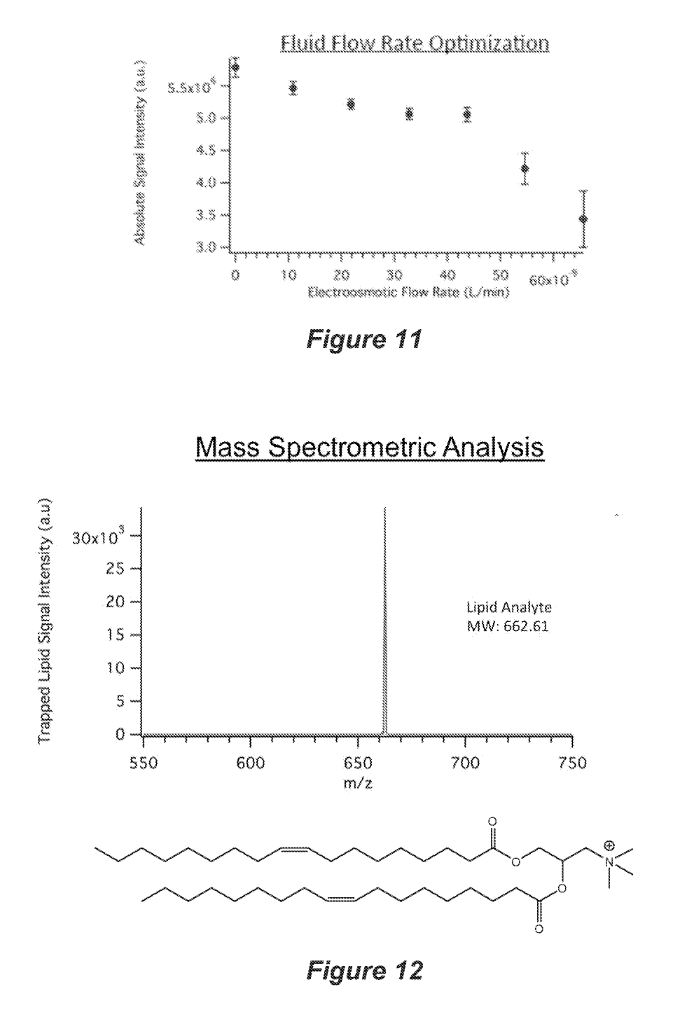

The capillary (21) is preferable made of a fused silica, to allow optimum interaction with the mass spectrometry device through the formation of a stable Taylor cone comprised of the solvent/biomarker mixture. The fused silica can be coated with a polymer or other suitable material to facilitate electrophoresis if needed, such as used in Successive Multiple Ionic Polymer Layer (SMIL) layers (Katayama et al., Anal. Chem. 1998, 70, 5272-5277). The 3-D microfluidic device, capillary and mass spectrometry devices should operate together at an optimum temperature and electroosmotic flow rate during electrophoresis to produce an optimum signal (see FIG. 10 and FIG. 11 as an example). FIG. 12 shows an example mass spectrometry reading when the 3-D microfluidic device and mass spectrometry device are coupled together.

The diameter of the capillary can be the same diameter of the main microchannel, or larger or smaller. In one embodiment, the diameter of the capillary is about 100 μM to about 250 μM, and more preferably about 150 μM to about 200 μM. The 3-D microfluidic device can also have an integrated emitter tip allowing coupling to a mass spectrometry device. The emitter tip can be made of a suitable material that is compatible with a non-aqueous solvent such as NMF.

### Method of Hydrophobic Biomarker Identification.

The invention provides a method of using the aforementioned 3-D microfluidic device to identify hydrophobic (e.g. lipid) biomarkers from a bodily fluid indicative of a disease state. The method generally comprises the steps of: 1) filing the 3-D microfluidic device with an appropriate non-aqueous solvent, 2) preparing a biological fluid sample for electrophoretic injection, 3) adding the sample to at least one sample loading microchannel, 4) applying a first voltage to the sample loading microchannel as to electrokinetically inject the sample through the NCAM, 5) applying a second voltage to the main microchannel to separate the sample into components, 6) injecting the separated samples into a mass spectrometer with a nanospray ionization source, and 7) identifying a lipid biomarker indicative of a disease state.

In some embodiments, the 3-D microfluidic device is filled with an electrophoresis solvent preferably a non-aqueous electrophoresis solvent. In some embodiments, the non-aqueous electrophoresis solvent is NMF. The solvent should be capable of solvating a hydrophobic biomarker without causing aggregation. In some embodiments, it may be desirable to add a background electrolyte to the electrophoresis solvent to ensure adequate electroosmotic flow of the sample. Tetraalkylammoniums salts produce excellent electroosmotic flow and are readily solvated by NMF. The concentration of background electrolyte can be experimentally determined. For instance, the concentration of tetraalkylammonium salts (e.g., TBA-TPhB or TPhP-TPhB) can be about 0 μm to about 10 mM.

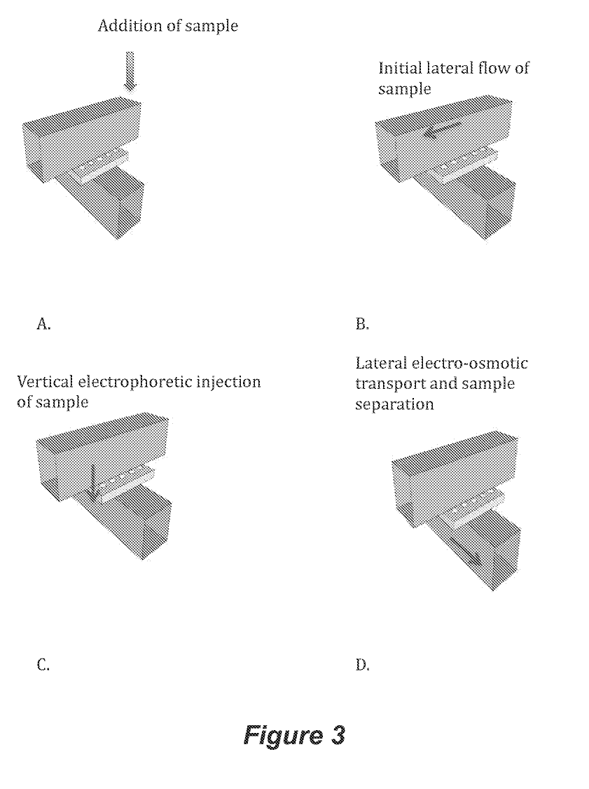

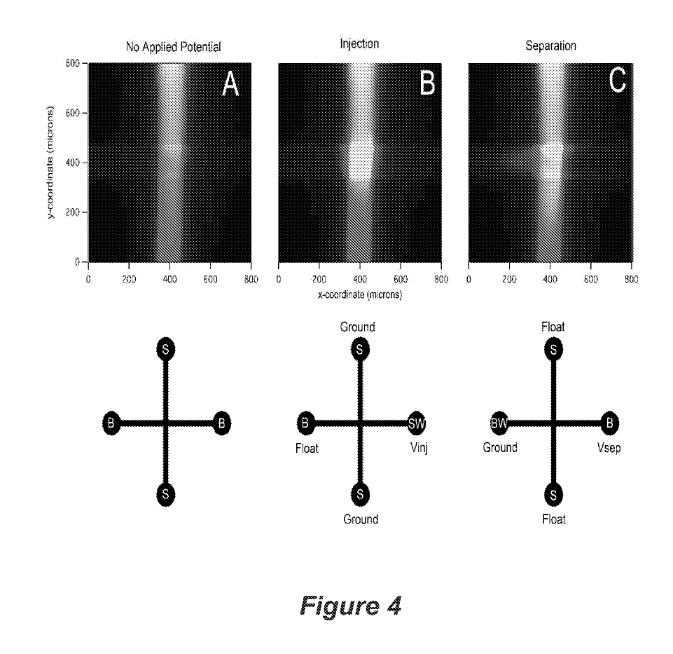

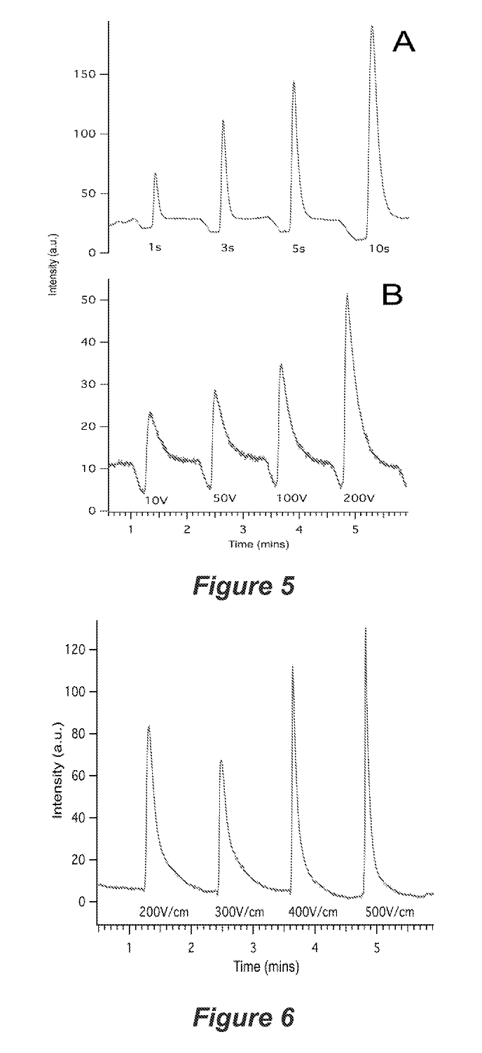

The biological fluid sample can then be added to the sample loading microchannel or a sample loading microchannel end as depicted in FIG. 3A. The sample can be added by manual injection, or through the use of an automated computer controlled device. As seen in FIGS. 3B and 4A, once the sample is added to the sample loading microchannel, it can occupy the length of the sample loading microchannel where no voltage is applied to the sample loading microchannel. The sample can then be electrokinetically injected by the application of an injection voltage where the electrodes at the sample loading microchannel endings are grounded while one electrode at a main microchannel ending is floated and a second electrode at the opposite main microchannel ending is active. This causes the movement of sample across the NCAM and into the main microchannel as depicted in FIGS. 3C and 4B. Preferably, 1 femtoliter or less of sample is electrokinetically injected. As shown in FIG. 5, both the length of time of the injection, as well as the voltage applied during the electrokinetic injection can affect the amount of sample injection. For instance, a longer the injection pulse and higher injection voltage allow for a larger sample amount to be injected into the main microchannel. The injection time can be about 1 second (s)-10 seconds (s), about 1 s-5 s, about 1 s-3 s, about 1 s or less than 1 s. In one aspect, the injection voltage is 1 s. Moreover, the injection voltage can be manipulated to allow various amounts of sample into the main microchannel. As an example, the injection voltage (V) can be about 10 V-200 V, about 10 V-100 V, about 10 V-50 V, about 10 V or less than 10 V. The injection time and injection voltage can manipulated independently of each other or in combination to fine tune the sample amount electrokinetically injected into the main microchannel.

After the sample has entered the main microchannel, a second voltage, or separation voltage is applied to the microfluidic device (see FIGS. 3D and 4C) where the electrodes in communication with the sample loading microchannel are floated and the electrode at the main microchannel ending previous floated is now grounded, driving the sample down the length of the main microchannel where it is separated into components. The separation voltage can be about 200 V cm⁻¹, or about 300 V cm⁻¹, or about 400 V cm⁻¹ or about 500 V cm⁻¹.

In some embodiments, the separated sample is coupled to a mass spectrometry device via a nanospray ionization source built into the device. The nanospray ionization source, for example, can be a fused silica capillary. The mass spectrometry device can be further coupled to a computer display to aid in identification of the lipid biomarkers.

The presence of some hydrophobic (e.g. lipid) biomarkers (e.g., prostaglandins, isoprostanes, etc.) can be indicative of a disease state. Moreover, elevated or reduced concentration of a lipid biomarker may be indicative of a disease state. The ratio of two or more lipid biomarkers can also be indicative of a disease state. The lipid biomarkers can also be used to monitor the progression of a disease state by monitoring the presence or concentrations of specific lipid biomarkers over a course of time. The number of lipid biomarkers separated during this method can be 1, 2, 3, 4, 5, 6, 7, 8, 9, or 10 or more.

As an example, isoprostanes type A-K, and in particular isoprostane type A, E, F, H and J are excellent candidates for lipid biomarkers.

In another example, a subgroup of F type isoprostanes, F₂-isoprostanes, were found at elevated levels in people afflicted with Multiple Sclerosis (Mattsson et al., 2007. Neurosci. Lett. 414(3), 233-236. (doi: 10.1016/J.Neulet.2006.12.044).

As a further example, another isoprostane type F2, has been used as a biomarker for Multiple Sclerosis (MS). One study examined the levels of isoprostane 8-epi-prostaglandin-F_{2α}(8-isoPG_{2α}) in individuals having Secondary Progressive MS (SPMS). Individuals with SPMS exhibited a four-fold, five-fold or six-fold higher concentration of 8-isoPG_{2α}(624 ng/mL) (SD=6.78) versus 93 ng/mL (SD=22.11) in a health controls group as found in urine samples (Miller et al., Neurochem Res. 2011 June; 36(6): 1012-1016.)

In another example, (8-isoPG_{2α}) was gound at significantly elevated levels in individuals with Rheumatoid Arthritis (ReA), Psoriatic Arthritis (PsA), Reactive Arthritis (RA) and Osteoarthritis (OA) as found in blood serum samples. ReA patients had an mean 8-isoPG_{2α} level of 451 (160 SEM) pg/mL, RA patients had 325 (143 SEM) pg/mL, 92 (226 SEM) pg/mL in PSA patients and 187 (53.3 SEM) pg/mL in patients with OA. The mean concentration in the health control sample exhibited 33 (3.3 SEM) pg/mL (Basu et al., Ann Rheum Dis 2001; 60:627-631).

Other examples of hydrophobic biomarkers include but are not limited to, hydrophobic polypeptides as biomarkers for renal disease, diabetes and sepsis; Prostate-specific-antigen (PSA) as a biomarker for prostate related disease; Carbohydrate-deficient transferin (CDT) as a biomarker for alcohol abuse; lowered levels of glusoseaminoglycans as biomarkers for some gastrointestinal carcinomas; hydrophobic amino acids as biomarkers for polyketonuria, maple syrup urine disease, histidinemia, COPD, pseudoxanthoma, cystinuria, and homocytinuria; nucleosides as biomarkers for thyroid cancer, breast cancer and leukemia; nucleic acid degradation products (8OHdg) as biomarkers for certain cancers; Polyclyclic aromatic hydrocarbons (PAH) as a biomarker for some cancers; hydroxytestosterone as a biomarker for breast cancer; uric acid, urea and creatine as biomarkers of gout, renal failure, leukemia and certain lymphomas and renal disease; thiocyanate as biomarker to smoking damage (Wittke et al., J. Chromatogr. A 2003, 1013, 173-181; Kaiser et al. Electrophoresis 2004, 25, 2044-2055; Weissinger et al., Kidney Int. 2004, 65, 2426-2434; Donohue et al., Anal. Biochem. 2005, 339, 318-327; Stiller et al., Clin. Chem. 1991, 37, 2029-2037; Legros et al., Clin. Chem. 2003, 49, 440-449; Wuyts et al., Clin. Chem. Lab. Med. 2003, 41, 739-746; Theocharis et al., Biomed. Chromatogr. 2002, 16, 157-161; 45] Qu et al., Clin. Chim. Acta 2001, 312, 153-162; Annovazzi et al., Electrophoresis 2004, 25, 683-691; Lochman et al., Electrophoresis 2003, 24, 1200-1207; La et al., Anal. Chim. Acta 2003, 486, 171-182; Liebich et al., J. Chromatogr. A 2005, 1071, 271-275; Burrows et al., Chem. Rev. 1998, 98, 1109-1151; Tagesson et al., Eur. J. Cancer, Part. A 1995, 31A, 934-940; Carrilho et al., J. Braz. Chem. Soc. 2005, 16, 220-226; Markushin et al., Chem. Res. Toxicol. 2003, 16, 1107-1117; Boughton et al., Electrophoresis 2002, 23, 3705-3710; Kong, et al., J. Chromatogr. A 2003, 987, 477-483; Valdes et al., Crit. Rev. Anal. Chem. 2004, 34, 9-23).

In some embodiments, the same 3-D microfluidic device and methods of use can be used for rapid screening of pharmaceutical products to ensure their active compounds are present at the appropriate quantities. In various embodiments, the presence of a biomarker can be indicative of a condition as described herein. In other embodiments, an increased level or concentration of a biomarker can be indicative of a condition as described herein. In yet further embodiments, a decreased level or concentration of a biomarker can be indicative of a condition as described herein. The increase or decrease can be, for example, at least about 10%, at least about 15%, at least about 20%, at least about 35%, at least about 50%, at least about 75%, at least about 100%, at least about 2-fold, at least about 3-fold, at least about 4-fold, at least about 5-fold, at least about 6-fold, at least about 7-fold, at least about 10-fold, or at least about 20-fold.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more peptides of a protein refers to one to five, or one to four, for example if the protein is fragmented.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

A “biofluid” refers to a fluid found in or derived from the body containing biological components including plasma, lipids, proteins, metabolites, combinations thereof, and the like.

A “biomarker” refers to a biomolecule found in a bodily fluid that is an indicator of a particular biological condition or process. A biomarker can be a lipid, a protein, a peptide, an amino acid, derivatives thereof, and the like.

A “lipid biomarker” refer to a lipid, a derivatives thereof, or a metabolite thereof, the presence of which, or the elevated or depressed level of which, is an indicator of a particular biological condition or process (e.g., a disease state described herein).

The term “lipid” refers to a hydrophobic or amphipathic small molecules that originate entirely or in part by carbanion-based condensations of thioesters and/or by carbocation-based condensations of isoprene units including fatty acyls, glycerolipids, glycerophospholipids, sphingolipids, saccharolipids, polyketides, sterol lipids and prenol lipids. Lipids include mono-, di- and triacylglycerols, phospholipids, free fatty acids, fatty alcohols, cholesterol, cholesterol esters, and the like. Lipids can be biomarkers that can be detected by the devices and methods described herein.

The term “phospholipid” as used herein refers to a glycerol phosphate with an organic headgroup such as choline, serine, ethanolamine or inositol and zero, one or two (typically one or two) fatty acids esterified to the glycerol backbone. Phospholipids that can be detected by the devices and methods described herein include, but are not limited to, phosphatidylcholine, phosphatidylethanolamine, phosphatidylserine and phosphatidylinositol as well as corresponding lysophospholipids. For example, a “phospholipid” can refer to an organic compound of Formula X:

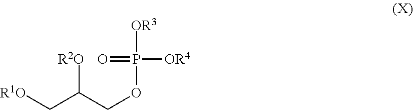

wherein R¹ is a fatty acid residue or H, R² is a fatty acid residue or H, R³ is H or a nitrogen containing compound such as choline (HOCH₂CH₂N⁺(CH₃)₃OH⁻), ethanolamine (HOCH₂CH₂NH₂), inositol, or serine, and R⁴ is a negative charge, H, or a cation such as an alkali metal cation (for example, Li⁺, Na⁺, or K⁺). In some embodiments, the nitrogen of ethanolamine can be acylated, for example, by acetate or by the acyl moiety of a fatty acid. In some embodiments, R¹ and R² are not simultaneously H. When R³ is H, the compound is a diacylglycerophosphate (also known as phosphatidic acid), while when R³ is a nitrogen-containing compound, the compound is a phosphatide such as lecithin, cephalin, phosphatidyl serine, or plasmalogen. The R1 site is referred to as position 1 of the phospholipid (per the stereospecific [sn] system of nomenclature), the R2 site is referred to as position 2 of the phospholipid (the sn2 position), and the R3 site is referred to as position 3 of the phospholipid (the sn3 position). Phospholipids also include phosphatidic acid and/or lysophosphatidic acid. Sphingolipids containing a phosphorus group are grossly classified as phospholipids; they contain a sphingosine base rather than a glycerol base.

A “microchannel” as used herein refers to a channel having a micron scale or smaller dimension, such as diameter, height, or width, of a cross-sectional profile. The “microchannel” can have a nano scale or smaller dimension, such as diameter, height, or width. Thus, a microchannel can have a cross-sectional dimension that is on the micron scale or smaller.

“Nanocapillary Array Membrane (NCAM)” as used herein refers to a nuclear etched membrane having a density of nanocapillaries extending through the membrane.

“Electropherogram” as used herein refers to a recording of the separated components of a mixture produced by electrophoresis by UV, UV-Vis absorption or laser-induced-fluorescence.

“Floating” or “floating the voltage” as used herein refers to electrically isolating an electrode connected to the device its power source such that the electrode assumes the potential value of the solution that it is in direct contact with.

“Aggregation” as used herein refers to a condition where a molecule preferential interacts with a like or similar molecule such that the molecule is no longer solvated.

“Solvating” as used herein refers to dissolving a target compound in a solvent whereby the target molecule spread out and become surrounded by solvent.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Examples

### Example 1

### Analysis of Lipid Biomarkers

In vivo measurements of lipid biomarkers are hampered by their low solubility in aqueous solution, which limits the choices for molecular separations. Here we introduce non-aqueous microchip electrophoretic separations of lipid mixtures performed in three-dimensional hybrid nanofluidic/microfluidic polymeric devices.

### 1. Introduction

Electrokinetic injection is used to reproducibly introduce discrete fL-pL volumes of charged lipids into a separation microchannel containing low (100 μM-10 mM) concentration tetraalkylammonium-tetraphenylborate background electrolyte in N-methylformamide, supporting rapid electroosmotic fluid flow in PDMS microchannels. The quality of the resulting electrophoretic separations depends on the voltage and timing of the injection pulse, the background electrolyte concentration, and the electric field strength. Injected volumes increase with longer injection pulse widths and higher injection pulse amplitudes. Separation efficiency, as measured by total plate number, N, increases with increasing electric field and with decreasing background electrolyte concentration. Electrophoretic separations of binary and ternary lipid mixtures were achieved with high resolution (Rₛ˜5) and quality (N>7.7×10⁶ plates m⁻¹). Rapid in vivo monitoring of lipid biomarkers requires high quality separation and detection of lipids downstream of microdialysis sample collection, and the multilayered non-aqueous microfluidic devices studied here offer one possible avenue to swiftly process complex lipid samples. The resulting capability may make it possible to correlate oxidative stress with in vivo lipid biomarker levels.

### 2. Materials and Methods

2.1 Reagents.

NBD-PA: 1-hexanoyl-2-[6-[(7-nitro-2-1,3-benzoxadiazol-4-yl)amino]-hexanoyl]-sn-glycero-3-phosphate (ammonium salt), NBD-PG: 1-hexanoyl-2-[6-[(7-nitro-2-1,3-benzoxadiazol-4-yl)amino]hexanoyl]-sn-glycero-3-[phospho-rac-(1-glycerol)] (ammonium salt), and NBD-CoA: [N-[(7-nitro-2-1,3-benzoxadiazol-4-yl)-methyl]amino] palmitoyl Coenzyme A (ammonium salt) were purchased from Avanti Polar Lipids Inc. (Alabaster, Ala., USA). TBA-TPhB: tetrabutylammonium tetraphenylborate, TPhP-TPhB: tetraphenylphosphonium tetraphenylborate, and N-methylformamide (NMF) were obtained from Sigma (St. Louis, Mo., USA). All were used without further purification.

2.2 BGE/Solvent Preparation.

Separation buffers were prepared from NMF solutions containing different (100 μM, 1 mM, and 10 mM) concentrations of either TBA-TPhB or TPhP-TPhB. Analyte solutions containing either NBD-PA, a mixture of NBD-PA and NBD-PG, or a mixture of NBD-PA, NBD-PG, and NBD-CoA were formulated in separation buffer at 1 nM, 10 nM, 1 μM, and 10 μM concentrations.

2.3 Microchip Fabrication.

Generally, the microchannels were aligned orthogonally with fluidic communication provided via the nanocapillary array membrane (NCAM) sandwiched between them. The assembled device, FIG. 1, consisted of four polymeric layers: two polydimethylsiloxane (PDMS) microchannel layers, one track-etched polycarbonate NCAM, and one PDMS adhesive layer. The adhesive layer effectively seals the device and prevents unwanted leakage. The master mold for microchannel fabrication was constructed by Stanford Microfluidics Foundry (Stanford, Calif., USA), and layers were produced using rapid prototyping. The sealing procedure for the device was adapted from the work of Chueh et al. (Anal. Chem. 2007, 79(9), 3504-3508).

Briefly, uncured PDMS was spun onto a glass cover slip for 1 min at 12,000 rpm. The thin uncured PDMS coating, on the order of tens of nanometers thick, was then stamped onto the top microchannel layer. The NCAM, purchased from Osmonics (Minnetonka, Minn., USA), was then positioned on the bottom microchannel layer just before both cured PDMS microchannel layers were brought into contact and pressed together firmly. Taking care to avoid pores located in the membrane area exposed to the orthogonal microchannels, the NCAM pores are filled with uncured PDMS. The device was then cured for 1 hour at 75° C. The microchannels were 100 μm in width and height. The source microchannel (top layer of FIG. 1) was 1.5 cm long and served as the sample reservoir. The receiving (separation) microchannel (bottom layer in FIG. 1) was 4.25 cm long. The 6-10 μm thick NCAM contained an array (4×10⁸ cm⁻²) of 100 nm diameter pores.

2.4 Instrumentation.

Fluidic control in the microchip was established using two high-voltage (HV) DC power supplies (602C-30P) from Spellman High Voltage Electronics Corp. (Hauppauge, N.Y., USA), specially constructed relay and switch boxes (University of Illinois, Urbana, Ill., USA), and a PCI data acquisition card (PCI-6221) from National Instruments (Austin, Tex., USA). A LabView (National Instruments) program controlled the voltage applied to each of the four Pd electrodes that drive electrokinetic flow. Analyte transport was observed using an Olympus IX-71 (Center Valley, Pa., USA) epifluorescence microscope featuring a 41001 fluorescein filter set (Chroma Technology Inc., Rockingham, Vt., USA). Illumination was obtained from a 100 W light source (X-Cite 120 PC) from Lumen Dynamics (Mississauga, ON, Canada). Images were recorded at 6 frames per second using a PhotonMax512 EMCCD camera (Princeton Instruments, Trenton, N.J., USA).

2.5 Procedures.

Fabricated devices were first vacuum-filled with solutions; analyte mixtures were loaded in the source microchannel, and separation buffer in the receiving microchannel. Microchips were then mounted onto the microscope stage where microfluidic microchannels were positioned above a 10× objective lens. Pd electrodes were placed in each of the four fluid reservoirs. Taking advantage of the transparency of PDMS, fluorescence intensity was observed along the length of the microfluidic microchannels. Following each 5-10 minute experiment, microchannels were rinsed with ca. 100 microchannel volumes of analyte solution or separation buffer by vacuum filling. Each of the experiments conducted in this work was performed in a new device. Although the magnitude of electroosmotic flow in these PDMS-based structures varied significantly across devices, relative analyte electrophoresis behavior remained consistent from device-to-device.

2.6 Solvent and Device Compatibility.

PDMS is known to swell in the presence of organic solvents. Chemically, NMF is closely related to DMF, a formamide that has been shown by previous work to swell PDMS minimally. Additionally, the PDMS layers and polycarbonate NCAMs showed no signs of degradation or chemical breakdown when left suspended in a bulk volume non-aqueous solvent for times as long as 48 hours.

### 3. Results and Discussion

3.1 Lipid Injections.

The spatially separated microchannels featured in this device can be bridged by an array of high aspect ratio nanocapillaries that simultaneously restrict free diffusion of analyte and facilitate electrokinetic injection. Just as with aqueous systems, relatively small potentials effect reproducible sample plug introduction. In separation experiments, a small (<1 nL) volume of fluorescently tagged lipids is first injected from the source microchannel, across the NCAM, into the separation microchannel and then transported downstream. The bias applied across the NCAM to achieve sample injection is defined by,

ΔV=V_{receiving}−V_{source} (1)

where V_{receiving} and V_{source} represent the relative potential of the separation microchannel and analyte reservoirs, respectively. FIG. 4 depicts how material is electrokinetically injected, where Vᵢₙⱼ and Vₛₑₚ represent the magnitude of the potential applied along the separation microchannel to drive either an injection or separation, respectively. A brief (t<1 s) voltage pulse across the NCAM electrophoretically injects lipid-containing solution into the separation microchannel. Floating the source microchannel electrodes then disengages the electric field across the NCAM, and a potential is applied along the length of the separation microchannel to complete the transfer of the injected sample into the separation region of the device via cation-driven electroosmotic flow (EOF) and begin the electrophoretic separation.

FIG. 5 shows the effect of the gate pulse duration, Δtᵢₙⱼ, and amplitude, ΔVᵢₙⱼ, on the quantity of material injected. Each peak represents a fluidic volume of material injected for a given time and then transported downstream (right to left in FIG. 4). The volumetric flow rate, F, of material injected through the NCAM can be written,

F=μ_{obs}\vec{E}ₐₚₚAₚₒᵣₑ (2)

where μ_{obs}, Eₐₚₚ, and Aₚₒᵣₑ represent the observed mobility, applied electric field, and effective cross sectional area, respectively. Based on the area beneath each peak, the data shown in FIG. 5 are consistent with Eqn. 2. Positive linear relationships are observed between both Δtᵢₙⱼ and ΔVᵢₙⱼ and the quantity of lipid transferred across the NCAM. The ability to tune the volume injected permits trade-offs between sensitivity and resolution. For example, for mass-limited samples, large values of Δtᵢₙⱼ and ΔVᵢₙⱼ can be used to enhance the sensitivity at the expense of a modest degradation in resolution. In addition, the reproducibility of injections depicted in FIG. 5 using a 3D hybrid architecture is commensurate with similar injections performed on aqueous systems.

3.2 Lipid Diffusion Coefficient.

The diffusion coefficient of the injected lipid molecules (Dₘ) determines the longitudinal dispersion of injected bands and thus can be employed to assess separation quality. Here Dₘ was calculated using the “on-the-fly-by-electrophoresis” method, according to,

$${D}_{m} = \frac{{( Δσ )}^{2}}{2 t} \qquad ( 3 )$$

where

Δσ=σ_{t>0}−σₜ₋₀ (4)

where σ represents the lipid bandwidth (μm), and t represents the time for the injected lipid packet to migrate from the injection to the observation point (400 μm). Although Eqn. 4 accounts for the finite width of the injected band, it does not account for the tailing (viz. FIG. 5), which is caused by electrical limitations of the high voltage supply. Currently, the minimum applied voltage (10 V) and application time (1 s) for electrokinetic injections are too high, resulting in significant injection on both sides of the NCAM, producing an apparent band tail. Improvements to incorporate a power supply that permits mV potentials and ms applications times are being implemented. The observed band tailing also influences interpretation of diffusion coefficients calculated based on Gaussian peak shapes, where based on analysis of repeated injections identical to those depicted in FIG. 3, (where t varies by ±7%) D_{m,NBD-PA}=4.48×10⁻⁷ cm² s⁻¹.

Band broadening, Δσ, increases with the square root of both Dₘ and t. Although this undesirable effect, which ultimately reduces resolution, is inevitable, the width of the injected band (σ_{τ=0}) is limited by the cross-sectional area (100 μm×100 μm) of the overlapping microchannels. Because separations are performed well above the concentration limit of detection (LOD), much smaller microchannels could be used which would result in further improvements in resolution. However, the need for a PDMS adhesion layer dictates that the microchannel width be sufficiently large to prevent blockage by uncured PDMS during assembly.

3.3 System Limit of Detection.

Robust biomarker detection in mammalian biofluids requires low LODs. To determine the LOD for the NAME experiment, discrete volumes of NBD-PA were injected into the separation microchannel at varying analyte concentration. The excitation source was set to maximum power (30 mW), and the resulting signal-to-noise (S/N) ratio of the fluorescence peak was measured downstream. Table 1 depicts the S/N for lipid concentrations in the range 100 pM<C<1 μM. Conservatively interpreting these data indicates that the LOD of the current system is ˜1-10 pM, a range acceptable for fluorescence-based assay development and easily competent for the determination of circulating plasma biomarkers. For example, mM lipid and protein levels and μM vitamin concentrations in plasma were used by Karaozene and coworkers to determine that obesity induces both oxidative stress and lipid composition changes in men. Conversely, lipid biomarkers for some conditions are present at much lower fM to pM concentrations in human biofluids. Picomolar concentrations of isoprostanes in plasma, for example, have been identified as early indicators of Rett syndrome. Addressing these more challenging biomarker assays in the NAME system described here will require improvements in LOD, physically concentrating the sample, or both.

TABLE 1
S/N Ratio as a Function of Concentration for NBD-PA Bands.

| NBD-PA | |
|---|---|
| Concentration | S/N^{a} |
| 100 pM | 31 ± 7 |
| 1 nM | 98 ± 33 |
| 10 nM | 175 ± 57 |
| 100 nM | 202 ± 83 |
| 1 μM | 358 ± 135 |

^{a}Measured 400 μm downstream in 10 mM TBA-TPhB/NMF.

3.4 Separation Performance Metrics.

3.4.a Plate number (N).

The number of theoretical plates is an indirect measure of the microchannel separation efficiency, indicating how well the system performs in the face of longitudinal diffusion and subsequent band broadening. N is defined by,

$$N = \frac{{μ}_{obs} {V}_{app} l}{2 {D}_{m} L} \qquad ( 5 )$$

where Vₐₚₚ=voltage applied across the separation microchannel, l=effective microchannel length, and L=total microchannel length over which voltage is applied. Plate numbers ranging from 10⁴-10⁵ are common in high quality electrophoretic separations.

3.4.b Background Electrolyte Concentration.

Table 2 shows the dependence of N on the ionic strength of the TBA-TPhB/NMF solution. Using a single device, TBA-TPhB/NMF solutions (100 μM, 1 mM, and 10 mM respectively) were introduced into both the analyte reservoir and the separation microchannel. Migrating peaks were then observed 400 μm downstream from the injection point, and the number of theoretical plates at 400 μm was determined by averaging the observed mobility values from several peaks produced at each electrolyte concentration. Qualitatively, EOF was observed to become less reproducible at the highest electrolyte concentrations. The thickness of the electrical double layer (κ⁻¹) for the TBA-TPhB/NMF separation media at the wall-solution interface is given by,

$${κ}^{- 1} = \sqrt{\frac{{ɛ}_{0} {ɛ}_{r} RT}{2 {F}^{2} {C}_{electrolyte}}} \qquad ( 6 )$$

where ∈ᵣ, ∈₀, R, T, F, and C_{electrolyte} represent the dielectric constant of the NMF solvent, permittivity of free space, gas constant, temperature, Faraday constant, and the TBA-TPhB concentration, respectively.

TABLE 2
Number of Theoretical Plates (N) vs. Ionic Strength of the BGE.

| TBATPhB Conc. | N^{a} |
|---|---|
| 100 μM | 538 ± 21 |
| 1 mM | 508 ± 61 |
| 10 mM | 428 ± 40 |

^{a}Lipid injections performed at ΔVᵢₙⱼ = 10 V, Δtᵢₙⱼ = 1 s, Eₛₑₚ = 424 V/cm, for each concentration. Measured 400 μm downstream.

Based on eqn. 6, κ⁻¹ for the concentrations investigated spans the range 5-50 nm. The dimensions of microchannels accommodating electrokinetic flow (on the order of 100 μm) are considerably larger than κ⁻¹, which indicates that the electrolyte concentration should not have a significant impact on the electroosmotic flow, and subsequent separation quality. However, Table 2 clearly shows a statistically significant improvement in the quality of separation with decreasing BGE concentration and is best when 100 μM TBA-TPhB/NMF is used. Furthermore, experiments have shown that the presence of TBA-TPhB at this concentration does not obviate analysis of NBD-PA using ambient ionization mass spectrometry.

3.4.c Separation Electric Field.

FIG. 6 shows the effect of electric field magnitude on band broadening in 100 μM TBA-TPhB. As shown, band broadening decreases with increasing electric field strength, and hence the efficiency of the lipid separation, as measured by N, improves at higher fields up to 500 V cm⁻¹. Although these findings suggest that optimal separations are achieved using the highest applied voltage the equipment permits, the maximum electric field is limited by Joule heating, which can decrease the viscosity of the separation media, promoting molecular diffusion and subsequent band broadening. In addition, fields in the 400-500 V cm⁻¹ range were sufficient to accomplish the separations of model compounds in these studies.

3.4.d Cation Hydrophobicity.

Due to its large dielectric constant (∈ᵣ=182), NMF exhibits excellent solvation properties for the TBA-TPhB background electrolyte chosen for these experiments. The association of the TBA cation with the negatively charged PDMS surface drives EOF in the presence of an applied field. In order to investigate how EOF affects performance of the NAME system, tetraphenylphosphonium, a more hydrophobic cation, was selected for comparison. Using NBD-PA, peak shapes in TBA-TPhB/NMF and TPhP-TPhB/NMF solutions were compared and it was determined that the tetraphenylphosphonium cation improves μ_{obs} of NBD-PA by ˜8%, a small, but statistically significant, effect.

3.5 NAME of Binary and Ternary Lipid Mixtures.

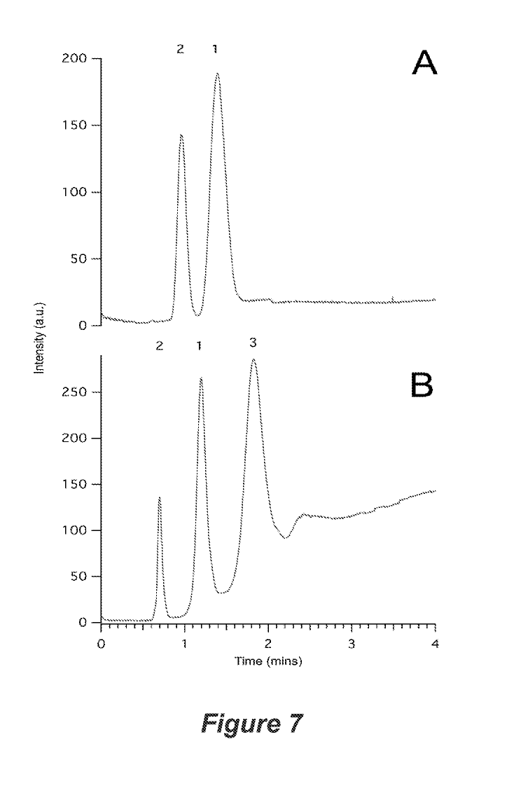

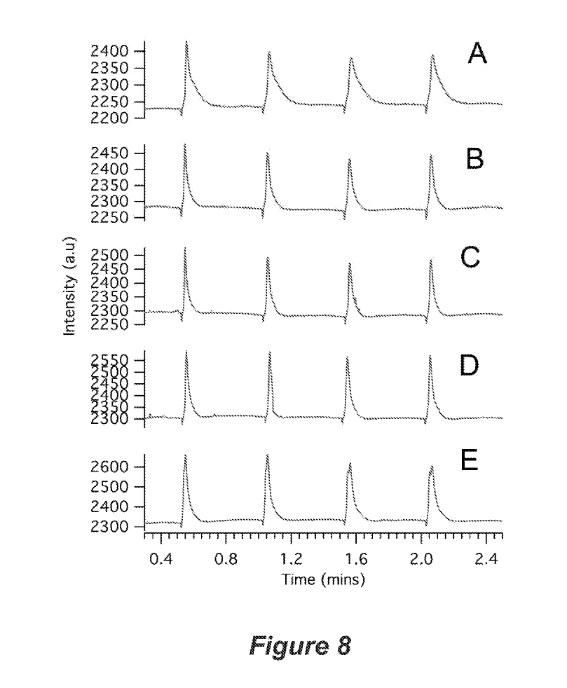

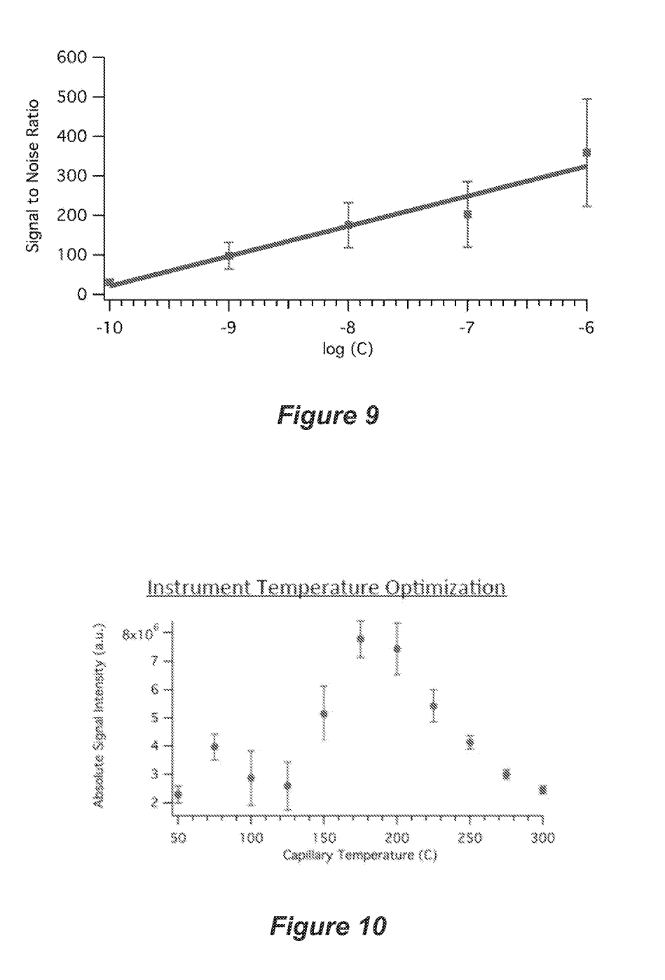

As shown in FIG. 7, fully resolved electrophoretic separations of both binary and ternary lipid mixtures are obtained 3.5 cm downstream of the injection point. In the case of cation driven electroosmotic flow, the electrophoretic driving force, determined by lipid charge-to-size ratio, opposes the bulk fluid motion. The order of migration of the three species agrees with predictions based on the molecular weights and charges of NBD-PA (MW=563.5, net charge=−1), NBD-PG (MW=637.6, net charge=−1), and NBD-CoA (MW=1234.4, net charge=−3), since NBD-CoA has the largest electrophoretic mobility (largest charge-to-size ratio), and NBD-PG has the smallest.

Another useful separation metric is the resolution, Rₛ, defined by Eqn. 7,

$${R}_{S , AB} = \frac{2 ( {t}_{A} - {t}_{B} )}{{w}_{A} + {w}_{B}} \qquad ( 7 )$$

where w represents the peak widths of the observed species. Acceptable baseline separation is achieved when Rₛ>1.5. The resolution in FIG. 5 for the binary separation is R_{s,21}=2.16, and in the case of the ternary separation, R_{s,21}=5.02 and R_{s,13}=3.48. The plate numbers for each species in the binary (N₁=1.65×10⁵, N₂=2.38×10⁵) and ternary (N₁=1.91×10⁵, N₂=3.26×10⁵, N₃=1.26×10⁵) separations are notable. However, peak capacity is relatively low, since it depends on the quantity of injected analyte, which is governed by the injection parameters (vide supra). Regardless, the combination of the initial separation and EOF results are sufficiently promising to vigorously pursue NAME as a viable on-site separation strategy for in vivo monitoring of lipid biomarkers.

### 4. Conclusions

Micromolar tetraalkylammonium salts in NMF constitute effective media for electrophoretic separations of intact lipids and their oxidation products. Further, these solutions are chemically compatible with low cost microchips that offer superb fluid control for precise handling and manipulation of analyte mixtures. Discrete lipid packets are controllably injected from a sample reservoir microchannel, across an NCAM, using low injection voltages (ΔVᵢₙⱼ<100 V) for Δtᵢₙⱼ=1-10 s. The sample voxels are introduced into a separation microchannel containing low ionic strength BGE, and, in the presence of sufficiently high applied electric fields, yield high resolution molecular separations of a quality comparable to those of commercial CE and NACE systems. In addition, relatively short separation microchannel lengths (roughly 1/10 the column length used in bench-top separation instruments) featured in this 3D architecture afford very rapid fluidic processing of lipid mixtures (typically <3 minutes).

Unlike time-consuming immunoassays, when NAME is coupled to an appropriate pre-processing strategy, such as microdialysis, it can rapidly separate and monitor lipid biomarkers obtained directly from patient biofluids. In addition to monitoring the levels of known biomarkers to track disease progression, NAME can be implemented in programs of biomarker discovery. Although separation performance in this work is assessed using fluorescence detection, the 3D NAME microchip can be coupled directly to a mass spectrometer (MS) for universal label-free detection. Softer ionization strategies promise to better maintain lipid integrity during analyte introduction into the MS, so current work in this laboratory is addressing the interfacing of NAME microchips to desorption electrospray ionization MS in order to combine this promising new approach to lipid separations and biomarker detection with highly sensitive label-free detection.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A microchip electrophoresis device comprising:
a substrate having at least a first layer, a second layer and a third layer;
the first layer having at least one main microchannel, the at least one main microchannel extending a first distance within the first layer, the at least one main microchannel having a first and second main microchannel endings;
the second layer having at least one sample loading microchannel, the at least one sample loading microchannel extending a second distance within the second layer, the at least one sample loading microchannel having a first and second sample loading microchannel endings where the at least one sample loading microchannel is transverse to the at least one main microchannel;
the third layer being a nanocapillary array membrane, the nanocapillary array membrane being disposed between and in fluid communication with the at least one main microchannel and the at least one sample loading microchannel;
a plurality of electrodes, the electrodes able to drive electrokinetic injection of the sample from the sample loading microchannel, through the nanocapillary array membrane, and into the main microchannel when a first voltage is applied; and the electrodes able to drive electrophoretic separation of the sample in the main microchannel when a second voltage is applied;
the microchip electrophoresis device being compatible with a non-aqueous solvent where the non-aqueous solvent is capable of solvating a hydrophobic biomarker without aggregation.

2. The device of claim 1, wherein at least one tertiary microchannel intersects the at least one main microchannel within the first layer.

3. The device of claim 1 comprising a plurality of main microchannels in the first layer.

4. The device of claim 1 containing a plurality of cross microchannels in the second layer the cross microchannels having a first end and a second end.

5. The device of claim 1 wherein the main microchannel is coupled to a mass spectrometry device.

6. The device of claim 2 wherein the at least one tertiary microchannel is coupled to a mass spectrometer.

7. The device of claim 1 wherein the nanocapillary array membrane is about 1-15 micrometers thick.

8. The device of claim 7 wherein the nanocapillary array membrane is about 6 to about 10 micrometers thick.

9. The device of claim 1 wherein the nanocapillary array contains pores that are about 10 nm to 10 μm in diameter.

10. The device of claim 9 wherein the nanocapillary array membrane contains pores that are about 90 nm to about 150 nm in diameter.

11. The device of claim 1 wherein the hydrophobic biomarker is a lipid.

12. A method of detecting a hydrophobic biomarker using the microchip electrophoresis device of claim 1, wherein the method comprises the steps of:
adding a non-aqueous solvent to the 3-D microfluidic device;
injecting a biofluid sample into a sample loading microchannel;
applying a first voltage to the sample loading microchannel so that the sample moves through the sample loading microchannel wherein the sample is electrokinetically injected through a nanocapillary array membrane into a main microchannel where the main microchannel, the sample loading microchannel and the nanocapillary array membrane are disposed in different layers but are in fluid communication;
floating the first voltage and applying a second voltage to the main microchannel where the sample is separated into components, the main microchannel being coupled to a detection device; and
analyzing the components with the detection device for the presence of a biomarker, the biomarker being a lipid or being derived therefrom, wherein the presence of the biomarker is indicative of a disease state.

13. A method for detecting lipid biomarkers using a 3-D microfluidic device, the method comprising:
adding a non-aqueous solvent to a 3-D microfluidic device;
injecting a biofluid sample into a sample loading microchannel;
applying a first voltage to the sample loading microchannel so that the sample moves through the sample loading microchannel wherein the sample is electrokinetically injected through a nanocapillary array membrane into a main microchannel where the main microchannel, the sample loading microchannel and the nanocapillary array membrane are disposed in different layers but are in fluid communication;
floating the first voltage and applying a second voltage to the main microchannel where the sample is separated into components, the main microchannel being coupled to a detection device; and
analyzing the components with the detection device for the presence of a biomarker, the biomarker being a lipid or being derived therefrom, wherein the presence of the biomarker is indicative of a disease state.

14. The method of claim 13 where the non-aqueous solvent comprises N-methyl formamide.

15. The method of claim 13 wherein the non-aqueous solvent contains at least one tetraalkylammonium salt.

16. The method of claim 13 wherein the detection of the sample is free of a synthetic label.

17. The method claim 13 wherein the detection device is a mass spectrometer.

18. The method of claim 13 wherein the lipid is an isoprostane.

19. The method of claim 13 wherein the lipid is isoprostane 8-epi-prostaglandin-F_{2α} wherein an elevated level of isoprostane 8-epi-prostaglandin-F_{2α} is at least two fold higher than the level of isoprostane 8-epi-prostaglandin-F_{2α} found in a healthy control sample, the two fold increase of isoprostane 8-epi-prostaglandin-F_{2α} being indicative a disease state.

20. The method of claim 19 wherein the disease state is that of Secondary Progressive Multiple Sclerosis.
